(19) United States
(12) Patent Application Publication
(10) Pub. No.: US 2013/0267465 A1
(43) Pub. Date: Oct. 10, 2013

(54) COLLAGENOUS MATERIAL

(71) Applicants: Paul Armitage, Leeds (GB); Christine Elizabeth Dawson, Leeds (GB)

(72) Inventors: Paul Armitage, Leeds (GB); Christine Elizabeth Dawson, Leeds (GB)

(73) Assignee: TISSUE SCIENCE LABORATORIES LIMITED, Leeds (GB)

(21) Appl. No.: 13/811,410

(22) PCT Filed: Jul. 21, 2011
(86) PCT No.: PCT/GB2011/051389

§ 371 Date: Jun. 24, 2013

(30) Foreign Application Priority Data
Jul. 22, 2010 (GB) 1012307.3

(51) Int. Cl. C07K 14/78

(52) U.S. Cl. CPC C07K 14/78
USPC 514/13.5; 435/273; 530/356

(57) ABSTRACT

A process for the manufacture of a collagenous material from a plurality of collagen particles is described. The collagen particles are derived from a natural tissue material, are substantially free of non-fibrous tissue proteins, cellular elements and lipids or lipid residues, and comprise fragments of collagen fibres displaying original collagen fibre architecture and molecular ultrastructure of the natural tissue material. The process comprises the step of freeze drying the collagen particles in suspension in a freeze-drying suspension medium.

The present invention relates to a collagenous material and to a process for the manufacture thereof. The collagenous material may be used, for example, in wound care.

Collagen is a triple-helix protein which forms the major part of the dermal extracellular matrix (ECM), together with glycosaminoglycans, proteoglycans, laminin, fibronectin, elastin and cellular components. The ECM is the largest component of the dermal skin layer and the synthesis of ECM is a key feature of wound healing, especially where there has been a significant loss of tissue that precludes closure by primary intention.

The principal function of collagen in the dermal ECM is to act as a scaffold in connective tissue. Predominantly, collagen is present in the form of type I collagen (80-85%) and type III collagen (8-11%), both of which are fibrillar or rod-shaped collagens. The tensile strength of skin is due largely to these collagen molecules assembling into fibrils, with adjacent molecules crosslinking to further increase tensile strength. However, collagen laid down during wound healing has a reduced structural integrity compared to unwounded tissue; scar tissue rarely exceeds 70% of unwounded tissue strength.

In addition to being the main component of scar tissue, collagen has a key role in the control of the inflammatory response to injury and subsequent repair, with functions that influence cellular mitogenesis, differentiation and migration; protein synthesis in the ECM; synthesis and release of inflammatory cytokines and growth factors; and interactions between enzymes which remodel the ECM, including matrix metalloproteinases (MMPs) and their tissue inhibitors (TIMPs).

For medical applications, purified fibrous collagen has long been known to be a useful therapeutic adjunct to aid wound healing. Research into collagen-based dressing materials has shown that collagen significantly increases the production of fibroblasts in wounds, encouraging direct migration of cells into the wound, enhancing the formation of new, organised collagen fibres.

Purified fibrous collagen pads have also been used to provide haemostatic dressings. When in contact with a bleeding surface, a fibrous collagen haemostat attracts platelets which adhere to collagen fibrils and release coagulation factors. This coagulation and aggregation of the platelets into thrombi on the collagenous mass, provides the formation of a physiologic platelet plug which slows and eventually stops the bleeding. The collagen fibrils also provide a structural matrix strengthening the platelet plug.

Previous studies have demonstrated that collagenous materials prepared using a process that retains the original fibre architecture and molecular ultrastructure of the natural tissues from which they are derived, have a number of advantageous properties. For example, U.S. Pat. No. 5,397,353 discloses a process for the preparation of collagenous materials that are substantially non-antigenic and substantially free of non-fibrous tissue proteins, cellular elements, lipids and lipid residues. The materials are typically sheet structures and are useful, for example, as implants. The collagenous sheet materials are susceptible to colonisation and vascularisation by host cells following implantation, and are resistant to calcification.

It has further been shown that the favourable properties of these collagen sheet materials can be retained when the collagen material is presented in the form of particles comprising fibre fragments. EP 1112096 describes an injectable or mouldable composition of collagen fibre fragments prepared from the relatively large-scale sheet materials of U.S. Pat. No. 5,397,353 in such a way as to retain the natural collagen fibre architecture and molecular ultrastructure. This size reduction is achieved by careful grinding or milling of the collagenous sheet materials, which may then be suspended to form a paste or injectable composition, as appropriate.

The present invention provides a new collagenous material particularly suitable for use as a wound dressing.

According to an aspect of the present invention there is provided a process for the manufacture of a collagenous material from a plurality of collagen particles, wherein said collagen particles are derived from a natural tissue material and are substantially free of non-fibrous tissue proteins, cellular elements and lipids or lipid residues, and wherein said collagen particles comprise fragments of collagen fibres displaying original collagen fibre architecture and molecular ultrastructure of said natural tissue material, said process comprising the step of freeze drying said collagen particles in suspension in a freeze-drying suspension medium.

Freeze drying the collagen particles gives rise to a coherent collagenous material. Surprisingly, the freeze-drying step does not result in a dry powder of collagen particles as would be expected. Unlike typical existing processes for manufacturing collagenous materials, the present invention does not require harsh chemical or mechanical treatment of the collagen starting material. The collagen particles themselves are initially prepared in such a way as to preserve original collagen fibre architecture and molecular ultrastructure of the natural tissue material from which they are derived, care being taken to avoid chemical or mechanical damage. Moreover, further processing of the collagen particles in the present invention avoids chemical or mechanical treatments that can damage the original collagen fibre architecture and molecular ultrastructure of the collagen fibre fragments making up the collagen particles. Thus, the collagenous material of the present invention comprises collagen displaying original collagen fibre architecture and molecular ultrastructure of the natural tissue material from which the collagen is derived.

In particular, in contrast to the known processes, the present invention does not require treatment of the collagen particles with acid to swell or solubilise the collagen. This is a significant advantage, since acid treatment is known to have a detrimental effect on the structure and integrity of the collagen fibres. Thus, particularly preferred embodiments of the present invention exclude treatment of the collagen particles with acid, in particular swelling of the collagen particles with acid.

Similarly, many prior art processes involve alkali treatment of collagen which is also known to be damaging to collagen. Thus, particularly preferred embodiments of the present invention exclude treatment of the collagen particles with alkali, in particular swelling of the collagen particles with alkali.

Thus, damage by acid or alkali swelling of collagen is avoided. Conditions for swelling collagen with acid and alkali are known in the art. As a guide, to minimise the risk of damage to the structure of the collagen, cross-linked collagen particles should preferably be maintained within a pH range of around 5 to 12, whereas non-cross-linked collagen particles should preferably be maintained within a pH range of about 6 to 8.

The present invention also avoids the need for extensive homogenisation associated with many existing processes, which can lead to mechanical damage to the collagen fibres. Thus, preferred embodiments of the present invention exclude homogenisation step(s).

The collagenous material has good properties for use as a wound dressing or as a component of a wound dressing. This porous, biocompatible material is non-antigenic and of natural origin, and provides a soft, sponge-like feel. The collagenous material is flexible, with a degree of conformability, which is clearly advantageous for application to wound sites. Significantly, the collagenous material has good cohesive strength and may be wrapped around wound areas to form an effective barrier. The collagenous material maintains a moist wound environment to promote wound healing, and its application helps to reduce contraction and scar tissue formation.

Being formed from a plurality of collagen particles, the collagenous material has a relatively uniform and consistent structure.

Further, the presence of collagen at a wound site is known to increase the rate of wound healing.

Thus, the collagenous material described herein is particularly useful in wound care. The collagenous material may be presented for use in a hydrated state. The process may therefore optionally further comprise at least one rehydration step, in which the collagenous material is maintained in an aqueous solution. For instance, the collagenous material may be maintained in saline, such as 0.9% saline.

The presentation of the collagenous material in a hydrated form helps to maintain a moist wound environment when the material is applied as a wound dressing. Unexpectedly, it has been found that the collagenous material formed from collagen particles remains intact following processing/manufacture and does not return to a particulate state following rehydration. Further, the collagenous material does not gel upon rehydration as seen with certain other wound dressings.

The coherent collagenous material produced using the process of the present invention may be manufactured in essentially any size, and can therefore be used to provide coverage of large wound sites, such as burns. The structural integrity of the collagenous material provides good protection to a wound site and the collagenous material may be readily secured in position to prevent migration.

The skilled person is aware that ‘freeze drying’, also referred to as ‘lyophilisation’, involves drying a material in a frozen state at very low pressure (i.e. in a high vacuum) so that ice, or other frozen solvent, sublimes rapidly without melting. Methods and apparatus for freeze drying are well known in the art (see, e.g., ‘Freeze-Drying/Lyophilization Of Pharmaceutical and Biological Products’, Third Edition, Louis Rey and Joan C. May, Informa Healthcare, 2010). The step of freeze drying may be carried out by any suitable method and using any suitable apparatus.

Any suitable freeze-drying suspension medium may be employed. For example, the freeze-drying suspension medium may comprise water or an aqueous medium, such as saline. Embodiments of the invention may exclude the use of freeze-drying suspension media containing collagen-swelling levels of acids or alkalis, in order to avoid damage to the fibre architecture and molecular ultrastructure of the collagen particles. By way of example, freeze-drying suspension media having a pH within the range of about 5 to 12 may be used to suspend cross-linked collagen particles, whereas for non-cross-linked collagen particles the freeze-drying suspension media may have a pH within the range of about 6 to 8.

The collagen particles may be suspended in the freeze-drying suspension medium at a range of different concentrations. Typically, the concentration of collagen particles suspended in the freeze-drying suspension medium is in the range of 10 to 80% (w/v), although other concentrations may be used. At the higher end of this range, the suspension of collagen particles in the freeze-drying medium has a relatively thick, pasty consistency.

The process may optionally include a step of mixing the suspension of collagen particles in the freeze-drying suspension medium. This ensures even distribution of the collagen particles in the freeze-drying suspension medium. Harsh mechanical mixing treatments such as homogenisation or blending are preferably avoided, however, in order to minimise collagen damage.

The natural tissue material may comprise any collagen-containing tissue material of human or animal (i.e. non-human) origin. The natural tissue material may be a tissue comprising predominantly type I collagen. Preferred starting materials include dermis and tendons. In some embodiments, it is preferred that porcine tissue materials are processed to provide the collagenous material, although it will be understood that other mammalian sources may alternatively be employed, such as, for example, primates, cows, sheep, goats or horses.

Depending upon the starting material, the particles of collagen may contain a proportion of elastin. Thus, the particles, and the coherent collagenous materials formed therefrom, consist essentially of collagen optionally with small proportions of elastin.

The particles of acellular collagen may be formed using any suitable process. The original collagen fibre architecture and molecular ultrastructure of the natural tissue material must be retained in the particles. Preferred processes for preparing the collagen particles are therefore analogous to those described in EP 1112096, the contents of which are incorporated herein by reference. Preferably, collagenous material prepared in the form of a sheet or other large-scale structure is milled to form the particles. The collagenous material may be prepared by a process analogous to those described in U.S. Pat. No. 5,397,353, the contents of which are incorporated herein by reference. The collagenous tissue is neither solubilised nor denatured in the process, so its natural fibre structure is maintained.

Thus, freshly cut natural tissue material may be treated to remove therefrom substantially all lipids and lipid residues and thereafter treated to remove non-fibrous tissue proteins and cellular elements.

The lipid extraction may be achieved by solvent extraction using an organic solvent, such as acetone. Other non-limiting examples of suitable solvents include non-aqueous solvents such as ethanol and ether.

Non-fibrous tissue proteins include glycoproteins, proteoglycans, globular proteins and the like, such as components of extracellular matrix. Cellular elements include antigenic proteins and enzymes and other cellular debris arising from the processing conditions. These portions of the natural tissue material may be removed by treatment with a proteolytic enzyme, such as trypsin. It has previously been found that above 20° C. treatment with trypsin can in some circumstances result in an alteration of the collagen fibre structure leading to a lower physical strength. Moreover, low temperatures discourage the growth of microorganisms in the preparation. It is therefore preferred to carry out the treatment with trypsin at a temperature below 20° C. Moreover, trypsin is more stable below 20° C. and lower amounts of it may be required. Any suitable trypsin concentration may be used, for instance a concentration within the range of around 0.01 g/l to 25 g/l. It has been found that good results can be obtained using about 2.5 g/l trypsin.

Further treatments may optionally be carried out, such as treatment with one or more additional enzymes, for example a carbohydrate-splitting enzyme.

Those substances said to be “substantially free” of materials generally contain less than 5% of, and preferably less than 1%, of said materials.

The resulting collagenous material is then reduced to particles, care being taken to ensure that the size reduction is not associated with a degradation of the original collagen fibre architecture and molecular ultrastructure of the starting material. The particles may be produced by grinding or milling using, for example, a ball or hammer mill, which may be cooled to an appropriate temperature. The sheet material may be cut into small pieces prior to milling. Milling may be carried out in dry form (less than 10% moisture content) or in frozen hydrated form (20-80% moisture content).

The collagen particles may be of any suitable size. Typically, the collagen particles have a mean diameter within the range of from around 5 μm to around 1000 μm, more typically from around 50 μm to around 500 μm. Good results have been achieved using collagen particles with a mean diameter of approximately 150 μm.

The freeze-dried collagenous material is porous, with a sponge-like appearance.

The process may optionally include a step of shaping or moulding the suspension of collagen particles to be freeze dried. Usefully, this allows the shape or conformation of the resulting freeze-dried collagenous material to be varied accordingly.

The suspension of collagen particles may, for example, be moulded, formed or cast into the desired shape prior to freeze-drying. Typically, the freeze-drying suspension medium and collagen particles suspended therein are held in a suitable mould during the freeze-drying step. The process can therefore be used for the manufacture of collagenous materials of predetermined shapes, which can readily be varied by using different moulds.

Non-limiting examples of suitable shapes include sheets, plugs, blocks, wedges, beads, ropes, or variations thereof.

Optionally, cross-linking may be carried out to impart additional physical strength to the collagenous material, and an increased resistance to digestive enzymes that may be present in a wound healing environment. Surprisingly, freeze-dried collagenous material formed from particles of cross-linked collagen has been found to have improved flexibility as compared to corresponding collagenous material formed from non-cross-linked collagen particles. This is beneficial for use of the collagenous material in wound care. Thus, preferred embodiments of the present invention include a step of cross-linking the collagen particles.

Whilst any cross-linking agent may be used, preferred cross-linking agents include polyisocyanates, in particular diisocyanates. The polyfunctional isocyanates react with amino or hydroxyl groups of different protein chains so forming a material which has a stable structure retaining the original architecture of the collagen and which is resistant to enzymatic attack. It is known that antigenicity is associated with the amino groups of the protein chains of collagen, and reacting the amino groups with isocyanate removes any antigenicity associated with these groups. Preferred diisocyantes include aliphatic, aromatic and alicyclic diisocyanates as exemplified by 1,6-hexamethylene diisocyanate, toluene diisocyanate, 4,4′-diphenylmethane diisocyanate, and 4,4′-dicyclohexylmethane diisocyanate, respectively. A particularly preferred diisocyanate is hexamethylene diisocyanate (HMDI). Carbodiimide cross-linking agents may also be used, such as 1-ethyl-3-[3-dimethylaminopropyl]carbodiimide hydrochloride (EDC).

The extent of cross-linking of the collagen particles may be varied. Usefully, this provides a mechanism for controlling the rate at which the collagenous material is resorbed or degraded during use. The resistance to degradation tends to increase as the extent of cross-linking is increased.

By way of example, cross-linking of the collagen particles may be carried out using HMDI. As a guide, the HMDI may be used at a concentration of around 0.01 g to 0.5 g per 50 g of collagen. If the concentration is too high, this may result in over-cross-linking and foreign body reactions. Cross-linking may be carried out for a range of different time periods. By way of example, the collagen may be exposed to the cross-linking agent for between around 1 hour and around 3 days. Typically, cross-linking is carried out for at least 12 hours, preferably at least 20 hours.

It will be appreciated that the cross-linking conditions may routinely be varied in order to adjust the extent of cross-linking.

According to a further aspect of the present invention there is provided a process for the manufacture of a collagenous material from a natural tissue material, wherein said process comprises steps of:
(i) treating the natural tissue material with an organic solvent; and
(ii) treating the natural tissue material with a proteolytic enzyme, so as to provide a treated material that is substantially free of non-fibrous tissue proteins, cellular elements and lipids or lipid residues and displays original collagen fibre architecture and molecular ultrastructure of the natural tissue material from which it is derived;
(iii) reducing said treated material to a plurality of collagen particles comprising fragments of collagen fibres; and
(iv) freeze drying said collagen particles in suspension in a freeze-drying suspension medium,
   wherein said process excludes swelling of the collagen particles with acid and/or alkali.

According to a further aspect of the invention there is provided a process for the manufacture of a collagenous material from a plurality of collagen particles, said collagen particles comprising fragments of collagen fibres and being derived from a treated material, said treated material having been produced from a natural tissue material by treatment with an organic solvent and with a proteolytic enzyme so as to provide a material that is substantially free of non-fibrous tissue proteins, cellular elements and lipids or lipid residues and displays original collagen fibre architecture and molecular ultrastructure of said natural tissue material, wherein said process comprises the step of freeze drying said collagen particles in suspension in a freeze-drying suspension medium and wherein said process excludes swelling of the collagen particles with acid and/or alkali.

According to a further aspect of the present invention there is provided a collagenous material obtainable by a process as herein described.

A range of different factors may be added to the collagenous material, such as growth factors, clotting agents, or other pharmaceutically active agents. The collagenous material may be seeded with cells, such as stem cells.

The collagenous material is useful in wound care. The collagenous material may also be used as a haemostat, to reduce or prevent blood flow from a body site.

According to a further aspect of the present invention there is provided a collagenous material as herein described for use in therapy.

According to a further aspect of the present invention there is provided the use in therapy of a collagenous material as herein described.

According to a further aspect of the present invention there is provided a collagenous material as herein described for use in wound care.

According to a further aspect of the present invention there is provided the use in wound care of a collagenous material as herein described.

According to a further aspect of the present invention there is provided a method of treatment of a wound comprising the step of applying to the wound a collagenous material as herein described.

According to a further aspect of the present invention there is provided a collagenous material as herein described for use as a haemostat.

According to a further aspect of the present invention there is provided the use in haemostasis of a collagenous material as herein described.

According to a further aspect of the present invention there is provided a method of reducing bleeding from a body site comprising the step of applying to the body site a collagenous material as herein described.

Embodiments of the present invention will now be described further in the following non-limiting examples.

### EXAMPLES

### 1. Manufacture of Freeze-Dried Collagenous Material

Acellular collagen sheet material was prepared according to the method disclosed in U.S. Pat. No. 5,397,353 and reduced to particles as described in EP 1112096.

Thus, freshly cut dermis harvested from sows was immersed in acetone. After 1 hour, the acetone was removed and replaced by fresh acetone. After a further incubation for around 36 hours, the tissue was removed from the acetone and placed in 0.9% saline to extract residual acetone. The tissue was then digested for 28 days with a solution of trypsin at a concentration of 2.5 mg/ml in 0.1M phosphate buffer with 0.5 mg/ml sodium azide as a bacteriostatic agent. The purified tissue was removed from the trypsin solution and rinsed in buffer.

The resulting collagenous sheet material was cut into small pieces (approximately 5×10 mm) before being milled cryogenically to a mean particle size of around 150 μm.

The collagen particles were then cross-linked using HMDI in acetone. A concentration of about 0.1 g HMDI per 25 g of solid collagen was added. The collagen was cross-linked for at least 20 hours and rinsed in acetone.

The cross-linked collagen particles were suspended in a freeze-drying suspension medium, being 0.9% saline. The suspension of collagen particles was placed in a polypropylene mould and freeze dried.

Freeze drying was carried out in a VirTis AdVantage Plus EL freeze-dryer (vacuum set point 600 mTorr; condenser set point −45° C.), using the freeze-drying cycle shown below.

Thermal treatment steps:

| Time (mins.) | Temp. (° C.) | Hold/Ramp |
|---|---|---|
| 1 | 20 | H |
| 110 | −35 | R |
| 70 | −35 | H |

Drying steps:

| Time (mins) | Temp. (° C.) | Hold/Ramp |
|---|---|---|
| 5 | −35 | H |
| 30 | −20 | R |
| 330 | −20 | H |
| 80 | 20 | R |
| 340 | 20 | H |

The resulting collagenous material was a flexible, sponge-like sheet having the general shape of the polypropylene mould. The sheet was porous, with a soft texture and had good physical integrity.

When rehydrated, the absorbent collagenous material quickly swelled. The collagenous material did not gel or fragment, and had good cohesive structural integrity.

### 2. Effect of Cross-Linking of the Collagen Particles

The process of Example 1 was repeated but the step of cross-linking the collagen particles with HMDI was omitted.

As with the collagenous material produced from cross-linked collagen particles, the collagenous material formed by freeze-drying non-cross-linked collagen particles also retained the general shape of the polypropylene mould. When rehydrated, neither material gelled or fragmented or returned to particulate form.

The collagenous material formed from cross-linked collagen particles was found to be softer and more flexible than the collagenous material formed from non-cross-linked collagen particles. Upon rehydration with saline, the former absorbed moisture more quickly and became more swollen. In rehydrated form, the collagenous material formed from cross-linked collagen particles had a sponge-like texture, whereas the collagenous material formed from non-cross-linked collagen particles was smoother and more dense.

It is of course to be understood that the invention is not intended to be restricted by the details of the above specific embodiments, which are provided by way of example only.

1-22. (canceled)

23. A process for the manufacture of a collagenous material from a natural tissue material, wherein said process comprises:
(i) treating the natural tissue material with an organic solvent;
(ii) treating the natural tissue material with a proteolytic enzyme, so as to provide a treated material that is substantially free of non-fibrous tissue proteins, cellular elements and lipids or lipid residues and displays original collagen fibre architecture and molecular ultrastructure of the natural tissue material from which it is derived;
(iii) reducing said treated material to a plurality of collagen particles comprising fragments of collagen fibres; and
(iv) freeze drying said collagen particles in suspension in a freeze-drying suspension medium, wherein said process excludes swelling of the collagen particles with acid and/or alkali.

24. The process according to claim 23, wherein the proteolytic enzyme is trypsin.

25. The process according to claim 23, wherein the process excludes homogenization of the collagen particles.

26. The process according to claim 23, wherein the process further includes a step of cross-linking the collagen particles.

27. The process according to claim 23, wherein the process further includes a step of shaping the suspension of collagen particles in the freeze-drying suspension medium.

28. The process according to claim 27, wherein the shaping of the suspension of collagen particles in the freeze-drying suspension medium is held in a mold for the step of freeze drying.

29. The process according to claim 23, wherein the process further includes a step of rehydrating the collagenous material.

30. The process according to claim 23, wherein the natural tissue material is of non-human origin.

31. A collagenous material comprising:
a plurality of freeze-dried collagen particles, wherein said collagen particles are derived from a natural tissue material and are substantially free of non-fibrous tissue proteins, cellular elements and lipids or lipid residues, and wherein said collagen particles comprise fragments of collagen fibres displaying original collagen fibre architecture and molecular ultrastructure of said natural tissue material, wherein the collagenous material does not include a dry powder of the collagen particles.

32. The collagenous material according to claim 31, wherein the material is selected from the group consisting of a sponge, a wound dressing, a hemostat and a barrier.

33. The collagenous material according to claim 31, wherein the plurality of collagen particles are cross-linked.

34. The collagenous material according to claim 31, wherein the natural tissue material is of non-human origin.

35. The collagenous material according to claim 31, wherein the material is porous.

36. A method of reducing bleeding from a body site comprising the step of applying to the body site a collagenous material according to claim 31.
